(19) United States
(12) Patent Application Publication
(10) Pub. No.: US 2017/0020571 A1
(43) Pub. Date: Jan. 26, 2017

(54) Additive manufacturing for spinal implants

(71) Applicant: Nexus Spine, LLC, Salt Lake City, UT (US)

(72) Inventors: David T. Hawkes, Pleasent Grove, UT (US); Peter Halverson, Draper, UT (US); Quentin Aten, Draper, UT (US)

(21) Appl. No.: 14/998,660

(22) Filed: Jan. 28, 2016

Related U.S. Application Data
(60) Provisional application No. 62/108,915, filed on Jan. 28, 2015.

(51) Int. Cl. A61B 17/70; B33Y 80/00; B33Y 10/00

(52) U.S. Cl. CPC A61B 17/705; B33Y 10/00; B33Y 80/00; A61B 17/7002; A61B 17/7032; A61B 17/7052; A61B 2017/00526

(57) ABSTRACT

Medical implants and implant components are formed by additive manufacturing processes. The additive manufacturing process used results in the implants or implant components having surfaces having a higher coefficient of friction as opposed to a similar implant manufactured using a different process, such as having a machined surface. The higher coefficient of friction of the relevant surface is particularly useful for multi-component implants that are to have a fixed relationship between the components based at least in part on a frictional engagement between them. While manufacturing via an additive manufacturing process may result in an implant component having slightly less strength for its size when compared with traditional manufacturing methods, the advantage of the increased coefficient of friction may offset any loss of component strength, and may allow for overall reduced implant size while maintaining other desirable implant characteristics.

### CROSS-REFERENCE TO RELATED APPLICATIONS

This application claims the benefit of U.S. Provisional Application No. 62/108,915, filed Jan. 28, 2015. This application is related to the following applications, all of which are incorporated herein by reference in their entireties: U.S. Ser. No. 11/952,709, filed Dec. 7, 2007 and entitled “Press-On Pedicle Screw Assembly,” U.S. Ser. No. 12/711,131 filed Feb. 23, 2010 and entitled “Press-On Link for Surgical Screws,” U.S. Ser. No. 13/455,854 filed Apr. 25, 2012 and entitled “Modular Construct System for Spinal Rod and Screw Assemblies,” and U.S. Ser. No. 14/060,757 filed Oct. 23, 2013 and entitled “Coupling System.” These related applications are referred to hereafter as the “Related Applications.”

### BACKGROUND OF THE INVENTION

1. Field of the Invention

The present invention relates to methods for manufacture of implants, and more particularly to systems and methods for additive manufacturing of implants.

2. Background and Related Art

As disclosed in the Related Applications, spinal fixation implants are often manufactured as having various components that are locked together during or after implantation so as to provide rigidity to the construct and assist with immobilization of one or more levels of the spine as spinal fusion occurs. Specific examples given in the Related Applications provide rigidity between the components of the implants by way of a press fit or interference fit achieved between the components. It would be desirable to be able to provide a more secure fit between the various components of an implant to reduce the possibility of unwanted motion occurring between implant components after implantation.

### BRIEF SUMMARY OF THE INVENTION

Implementations of the invention provide medical implants and implant components formed by additive manufacturing processes. The additive manufacturing process used results in the implants or implant components having surfaces having a higher coefficient of friction as opposed to a similar implant manufactured using a different process, such as a machined surface. The higher coefficient of friction of the relevant surface is particularly useful for multi-component implants that are to have a fixed relationship between the components based at least in part on a frictional engagement between them, such as in the devices disclosed in the Related Applications. While manufacturing via an additive manufacturing process may result in an implant component having slightly less strength than if the component were manufactured using traditional processes such as machining from bulk material, the advantage of the increased coefficient of friction may offset any loss of component strength, and may allow for overall reduced implant size while maintaining other desirable implant characteristics.

According to certain implementations of the invention, a method for manufacturing an implant includes steps of using an additive manufacturing process to create a first implant component from a bulk material, the first implant component having a contacting surface adapted to contact a second implant component upon implantation, wherein the additive manufacturing process used causes the contacting surface to have a higher coefficient of friction than a coefficient of friction of a machined surface of a similar material not made using an additive manufacturing process. The first implant component may be a pedicle screw. The contacting surface may be a surface of a ball head of the pedicle screw.

Alternatively, the first implant component may be a tulip assembly adapted to engage and secure a pedicle screw. The contacting surface may include an inner surface of a cavity adapted to receive a head of a pedicle screw therein. The contacting surface may include an inner surface of a cavity adapted to receive a connecting rod therein. The contacting surface may include an inner threaded surface adapted to receive a set screw therein, or a surface of the set screw.

The additive manufacturing process may be a process using a material such as commercially pure titanium or a titanium alloy. The additive manufacturing process may be a process such as electron beam melting, selective laser sintering, direct metal laser sintering, selective laser melting, laser metal deposition-wire, and electron beam freeform fabrication.

According to further implementations of the invention, a multi-component medical implant may include a first component formed of a material and having a contact surface at least partially formed by an additive manufacturing process and a second component having a contact surface adapted to contact and fixedly engage the contact surface of the first component. After implantation the first component and the second component are at least partially fixed relative to each other due to a frictional engagement between and at their respective contact surfaces. The contact surface of the first component has a higher coefficient of friction as compared to a machined surface of a similar material not made by an additive manufacturing process.

The first and second components may be spinal fixation implant components such as a pedicle screw, a tulip assembly, or a tulip-to-tulip interconnecting rod. The first component may be entirely formed by the additive manufacturing process. The second component's contact surface may be at least partially formed by the additive manufacturing process, or the second component may be entirely formed by the additive manufacturing process.

The additive manufacturing process may be a process such as electron beam melting, selective laser sintering, direct metal laser sintering, selective laser melting, laser metal deposition-wire, and electron beam freeform fabrication. The additive manufacturing process may be a process using a material such as commercially pure titanium or a titanium alloy.

The contact surface of the first component may include a surface such as an outer surface of a head of a pedicle screw, an inner surface of a cavity of a tulip assembly adapted to engage an outer surface of a head of a pedicle screw, a surface of a cavity of a tulip assembly adapted to engage a rod interconnecting tulip assemblies, a surface of a rod interconnecting tulip assemblies, a threaded surface of a tulip assembly adapted to receive a set screw, or a threaded surface of a set screw adapted to engage a threaded surface of a tulip assembly. The contact surface of the first component and the contact surface of the second component may be adapted to engage each other via a press fit.

According to further implementations of the invention, a multi-component spinal fixation implant includes a first component formed by an additive manufacturing process and comprising a ball end defined by a partially spherical outer surface and a second component formed by an additive manufacturing process and comprising a cavity adapted to receive the ball end of the first component and to engage the ball end of the first component via a press fit. After implantation the first component and the second component may be at least partially fixed relative to each other due to a frictional engagement of the press fit between the ball end and the cavity. The surfaces of the ball end and of the cavity may have a higher coefficient of friction as compared to a machined surface of a similar material not made by an additive manufacturing process. The first and second components may be spinal fixation implant components such as a pedicle screw, aa tulip assembly, or a tulip-to-tulip interconnecting rod.

### BRIEF DESCRIPTION OF THE SEVERAL VIEWS OF THE DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows a representative implant which may be made using an additive manufacturing process;

FIG. 2 shows another representative implant which may be made using an additive manufacturing process;

FIG. 3 shows another representative implant which may be made using an additive manufacturing process; and

FIG. 4 shows another representative implant which may be made using an additive manufacturing process.

### DETAILED DESCRIPTION OF THE INVENTION

A description of embodiments of the present invention will now be given with reference to the Figures. It is expected that the present invention may take many other forms and shapes, hence the following disclosure is intended to be illustrative and not limiting, and the scope of the invention should be determined by reference to the appended claims.

In the specification and in the claims, the term “similar material” is intended to mean a material having commercially identical chemical composition. Thus, a material or device manufactured via an additive manufacturing process might have differing structural characteristics than a material or device manufactured using a different process, but if the material or device were broken down into its base components, the base components would be commercially identical.

Embodiments of the invention provide medical implants and implant components formed by additive manufacturing processes. The additive manufacturing process used results in the implants or implant components having surfaces having a higher coefficient of friction as opposed to a similar implant manufactured using a different process, such as a machined surface. The higher coefficient of friction of the relevant surface is particularly useful for multi-component implants that are to have a fixed relationship between the components based at least in part on a frictional engagement between them, such as in the devices disclosed in the Related Applications. While manufacturing via an additive manufacturing process may result in an implant component having slightly less strength than a similarly sized implant manufactured using traditional methods, the advantage of the increased coefficient of friction may offset any loss of component strength, and may allow for overall reduced implant size while maintaining other desirable implant characteristics.

According to certain embodiments of the invention, a method for manufacturing an implant includes steps of using an additive manufacturing process to create a first implant component from a bulk material, the first implant component having a contacting surface adapted to contact a second implant component upon implantation, wherein the additive manufacturing process used causes the contacting surface to have a higher coefficient of friction than a coefficient of friction of a machined surface of a similar material not made using an additive manufacturing process. The first implant component may be a pedicle screw. The contacting surface may be a surface of a ball head of the pedicle screw.

Alternatively, the first implant component may be a tulip assembly adapted to engage and secure a pedicle screw. The contacting surface may include an inner surface of a cavity adapted to receive a head of a pedicle screw therein. The contacting surface may include an inner surface of a cavity adapted to receive a connecting rod therein. The contacting surface may include an inner threaded surface adapted to receive a set screw therein, or a surface of the set screw.

The additive manufacturing process may be a process using a material such as commercially pure titanium or a titanium alloy. The additive manufacturing process may be a process such as electron beam melting, selective laser sintering, direct metal laser sintering, selective laser melting, laser metal deposition-wire, and electron beam freeform fabrication.

According to further embodiments of the invention, a multi-component medical implant may include a first component formed of a material and having a contact surface at least partially formed by an additive manufacturing process and a second component having a contact surface adapted to contact and fixedly engage the contact surface of the first component. After implantation the first component and the second component are at least partially fixed relative to each other due to a frictional engagement between and at their respective contact surfaces. The contact surface of the first component has a higher coefficient of friction as compared to a machined surface of a similar material not made by an additive manufacturing process.

The first and second components may be spinal fixation implant components such as a pedicle screw, a tulip assembly, or a tulip-to-tulip interconnecting rod. The first component may be entirely formed by the additive manufacturing process. The second component's contact surface may be at least partially formed by the additive manufacturing process, or the second component may be entirely formed by the additive manufacturing process.

The additive manufacturing process may be a process such as electron beam melting, selective laser sintering, direct metal laser sintering, selective laser melting, laser metal deposition-wire, and electron beam freeform fabrication. The additive manufacturing process may be a process using a material such as commercially pure titanium or a titanium alloy.

The contact surface of the first component may include a surface such as an outer surface of a head of a pedicle screw, an inner surface of a cavity of a tulip assembly adapted to engage an outer surface of a head of a pedicle screw, a surface of a cavity of a tulip assembly adapted to engage a rod interconnecting tulip assemblies, a surface of a rod interconnecting tulip assemblies, a threaded surface of a tulip assembly adapted to receive a set screw, or a threaded surface of a set screw adapted to engage a threaded surface of a tulip assembly. The contact surface of the first component and the contact surface of the second component may be adapted to engage each other via a press fit.

According to further embodiments of the invention, a multi-component spinal fixation implant includes a first component formed by an additive manufacturing process and comprising a ball end defined by a partially spherical outer surface and a second component formed by an additive manufacturing process and comprising a cavity adapted to receive the ball end of the first component and to engage the ball end of the first component via a press fit. After implantation the first component and the second component may be at least partially fixed relative to each other due to a frictional engagement of the press fit between the ball end and the cavity. The surfaces of the ball end and of the cavity may have a higher coefficient of friction as compared to a machined surface of a similar material not made by an additive manufacturing process. The first and second components may be spinal fixation implant components such as a pedicle screw, aa tulip assembly, or a tulip-to-tulip interconnecting rod.

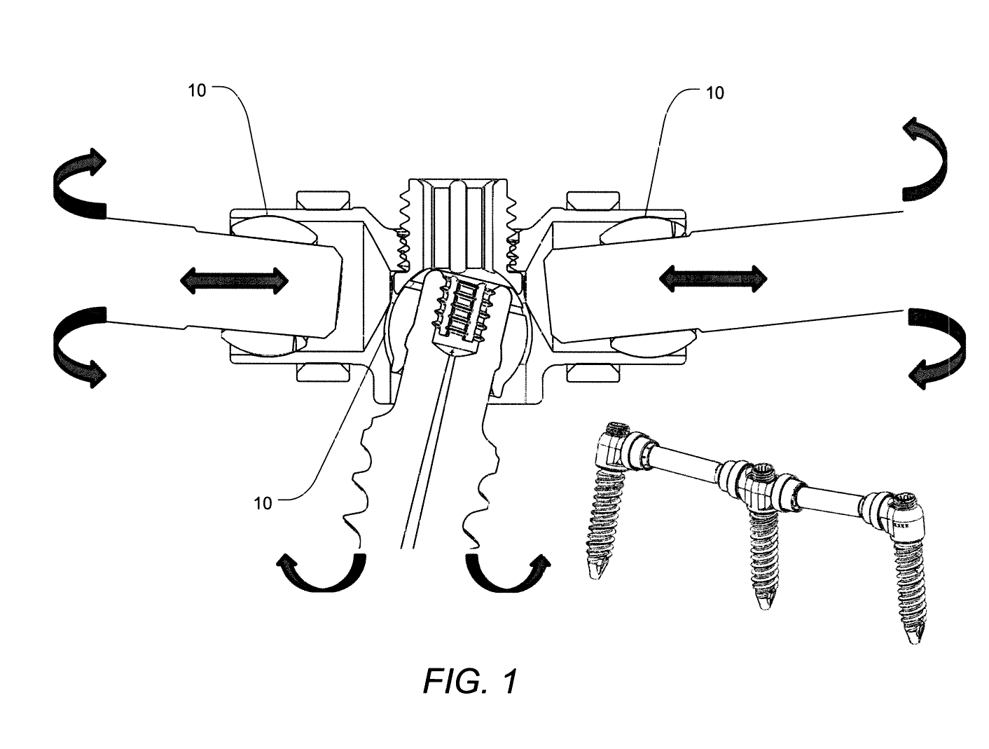

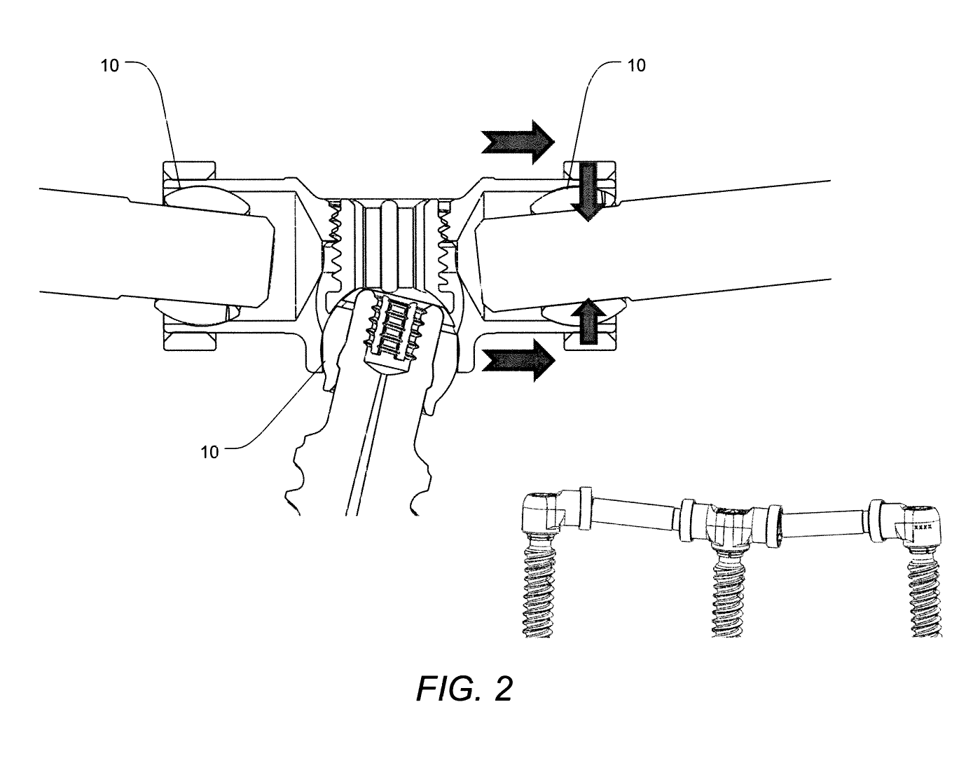

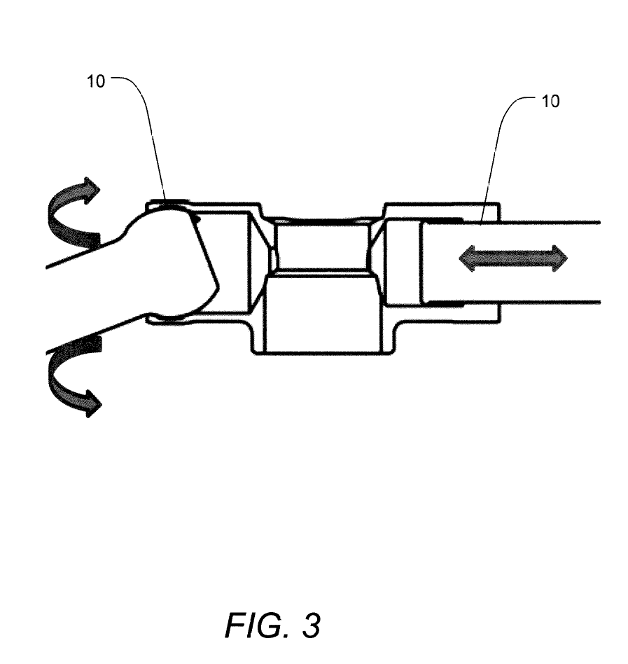

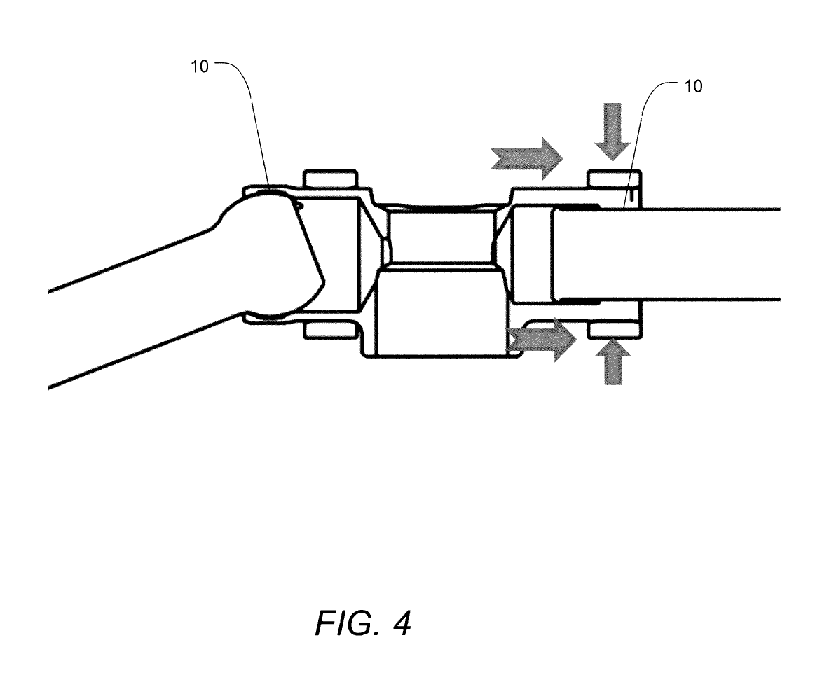

FIGS. 1-4 show various illustrative multi-component spinal implants, showing various connections 10 between components of the implants. The connections 10 between the components of each implant are connections that allow for polyaxial and/or translational movement between the components before the components are engaged together via a press fit or interference fit as described in the Related Applications. Once the press fit or interference fit is generated between the components, the components are prevented from movement relative to one another, as is described in more detail in the Related Applications.

Of primary import for this discussion is the fact that manufacturing of the components of the implant via an additive manufacturing process (otherwise known as 3D printing) has a benefit of producing a rough surface of the components at the locations of the press fit or interference fit without the need for post processing to achieve the rough surface. Other methods for generating a rough surface may include chemical, mechanical, or laser/electron beam methods. Providing a roughened surface at least at the locations of the press fit or interference fit increases the coefficient of friction at those locations as compared to an implant made of a similar material but via machining.

The increased coefficient of friction increases the locking strength of the press fit or interference fit. This increase in strength of the press fit or interference fit may be achieved without increasing the size of the implant, so the amount of implanted material inside of the body of the patient may be reduced. Additionally, even though manufacturing an implant via an additive manufacturing process may result in a device that is somewhat weaker than a similarly sized device made from a similar material, the increased strength of locking may offset any concomitant reduction in the strength of the implant components themselves. Embodiments of the invention allow for the manufacturing of implants of increased strength without increasing the manufacturing costs or size of the implant.

Implants may be manufactured using any desirable bio-compatible material that is also compatible with a selected additive manufacturing process and that has the desired strength characteristics. By way of example only, such materials may include commercially pure titanium as well as titanium alloys. Those of ordinary skill in the implant and additive manufacturing arts will be able to select appropriate materials and manufacturing processes from currently available materials and processes, as well as any materials or processes invented in the future using routine experimentation. Also by way of example only, exemplary additive manufacturing processes that could be used to make embodiments of implants might include processes such as electron beam melting, selective laser sintering, direct metal laser sintering, selective laser melting, laser metal deposition-wire, and electron beam freeform fabrication. Of course, not all additive manufacturing processes may be appropriate for every desired material and every desired implant characteristic.

While embodiments of the invention have been described with specific reference to multi-component spinal implants that are connectable via a press fit or interference fit as described in the Related Applications, it is envisioned that the use of additive manufacturing processes to manufacture spinal implants may provide similar higher-coefficient-of-friction advantages to predicate devices such as those devices discussed in the background sections of the Related Applications. As such, use of additive manufacturing processes as described herein is embraced as additional illustrative embodiments of the invention to form traditional spinal fixation implants with separate tulip heads using set screws to secure physician-bent interconnecting rods between pedicle screws. Additive manufacturing may provide improvements to the security of any desired component of such devices, such as to the pedicle screw, the tulip head or assembly, the set screws, the interconnecting rods, any applicable spacers, etc.

Similarly, while embodiments of the invention have been discussed primarily with respect to spinal implants, similar benefits may accrue to any multi-component implant for use in any portion of the body. Embodiments of the invention include the use of components at least partially made using an additive manufacturing process so as to achieve an engaging surface having a higher coefficient of friction, such that a more secure fixed relationship can be achieved between the components of the implant when desired. Thus, embodiments of the invention are not necessarily limited to the area of spinal implants or spinal fixation implants.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. A method for manufacturing an implant comprising:
using an additive manufacturing process to create a first implant component from a material, the first implant component having a contacting surface adapted to contact a second implant component upon implantation, wherein the additive manufacturing process used causes the contacting surface to have a higher coefficient of friction than a coefficient of friction of a machined surface of a similar material not made using an additive manufacturing process.

2. The method for manufacturing as recited in claim 1, wherein the first implant component comprises a pedicle screw.

3. The method for manufacturing as recited in claim 2, wherein the contacting surface comprises a surface of a ball head of the pedicle screw.

4. The method for manufacturing as recited in claim 1, wherein the first implant component comprises a tulip assembly adapted to engage and secure a pedicle screw.

5. The method for manufacturing as recited in claim 4, wherein the contacting surface comprises an inner surface of a cavity adapted to receive a head of a pedicle screw therein.

6. The method for manufacturing as recited in claim 4, wherein the contacting surface comprises an inner surface of a cavity adapted to receive a connecting rod therein.

7. The method for manufacturing as recited in claim 4, wherein the contacting surface comprises an inner threaded surface adapted to receive a set screw therein.

8. The method for manufacturing as recited in claim 1, wherein the additive manufacturing process comprises a process using a material selected from the group consisting of commercially pure titanium and a titanium alloy.

9. The method for manufacturing as recited in claim 8, wherein the additive manufacturing process comprises a process selected from the group consisting of:
electron beam melting;
selective laser sintering;
direct metal laser sintering;
selective laser melting;
laser metal deposition-wire; and
electron beam freeform fabrication.

10. A multi-component medical implant comprising:
a first component formed of a material and having a contact surface at least partially formed by an additive manufacturing process; and
a second component having a contact surface adapted to contact and fixedly engage the contact surface of the first component;
wherein after implantation the first component and the second component are at least partially fixed relative to each other due to a frictional engagement between and at their respective contact surfaces, and wherein the contact surface of the first component has a higher coefficient of friction as compared to a machined surface of a similar material not made by an additive manufacturing process.

11. The multi-component medical implant as recited in claim 10, wherein the first and second components comprise spinal fixation implant components selected from the group consisting of:
a pedicle screw;
a tulip assembly; and
a tulip-to-tulip interconnecting rod.

12. The multi-component medical implant as recited in claim 10, wherein the first component is entirely formed by the additive manufacturing process.

13. The multi-component medical implant as recited in claim 12, wherein the second component's contact surface is at least partially formed by the additive manufacturing process.

14. The multi-component medical implant as recited in claim 13, wherein the second component is entirely formed by the additive manufacturing process.

15. The multi-component medical implant as recited in claim 10, wherein the additive manufacturing process comprises a process selected from the group consisting of:
electron beam melting;
selective laser sintering;
direct metal laser sintering;
selective laser melting;
laser metal deposition-wire; and
electron beam freeform fabrication.

16. The multi-component medical implant as recited in claim 10, wherein the contact surface of the first component comprises a surface selected from the group consisting of:
an outer surface of a head of a pedicle screw;
an inner surface of a cavity of a tulip assembly adapted to engage an outer surface of a head of a pedicle screw;
a surface of a cavity of a tulip assembly adapted to engage a rod interconnecting tulip assemblies;
a surface of a rod interconnecting tulip assemblies;
a threaded surface of a tulip assembly adapted to receive a set screw; and
a threaded surface of a set screw adapted to engage a threaded surface of a tulip assembly.

17. The multi-component medical implant as recited in claim 10, wherein the additive manufacturing process comprises a process using a material selected from the group consisting of commercially pure titanium and a titanium alloy.

18. The multi-component medical implant as recited in claim 10, wherein the contact surface of the first component and the contact surface of the second component are adapted to engage each other via a press fit.

19. A multi-component spinal fixation implant comprising:
a first component formed from a material by an additive manufacturing process and comprising a ball end defined by a partially spherical outer surface; and
a second component formed from the material by an additive manufacturing process and comprising a cavity adapted to receive the ball end of the first component and to engage the ball end of the first component via a press fit;
wherein after implantation the first component and the second component are at least partially fixed relative to each other due to a frictional engagement of the press fit between the ball end and the cavity, and wherein the surfaces of the ball end and of the cavity have a higher coefficient of friction as compared to a machined surface of a similar material not made by an additive manufacturing process.

20. The multi-component spinal fixation implant as recited in claim 19, wherein the first and second components comprise spinal fixation implant components selected from the group consisting of:
a pedicle screw;
a tulip assembly; and
a tulip-to-tulip interconnecting rod.
